(19) United States
(12) Patent Application Publication
(10) Pub. No.: US 2003/0236446 A1
(43) Pub. Date: Dec. 25, 2003

(54) Endoscope device

(76) Inventor: Teruo Eino, Tokyo (JP)

Correspondence Address: DICKSTEIN SHAPIRO MORIN & OSHINSKY LLP, 1177 AVENUE OF THE AMERICAS (6TH AVENUE), 41 ST FL., NEW YORK, NY, 10036-2714, US

(21) Appl. No.: 10/462,678

(22) Filed: Jun. 17, 2003

(30) Foreign Application Priority Data
Jun. 21, 2002 (JP) 2002-181910

(51) Int. Cl. A61B 1/06

(52) U.S. Cl. 600/160

(57) ABSTRACT

The endoscope device comprises an insert portion, which has a solid-state image pickup element in the distal end portion thereof and which is inserted in an examination target; a device main body, which houses a light source that supplies illumination light to a light guide of the insert portion, a CCU for processing an image pickup signal picked up by the solid-state image pickup element of the insert portion, and an A/D converter for converting an image signal into a digital image signal, the device main body being connected to the insert portion; and a general-purpose PDA which is freely detachably connected to the device main body and which comprises a memory card for recording the image signal thus converted into a digital signal, and a monitor for displaying images, and the like.

This application claims benefit of Japanese Patent Application No. 2002-181910 filed in Japan on Jun. 21, 2002, the contents of which are incorporated by this reference.

### BACKGROUND OF THE INVENTION

1. Field of the Invention

The present invention relates to an endoscope device that comprises an insert portion and performs examinations of a variety of internal parts.

2. Description of the Related Art

In recent years, endoscopes have come to be adopted widely in medical fields and industrial fields and so forth.

A variety of such endoscopes have been proposed in U.S. Pat. Nos. 4,803,562, 5,153,721, 6,120,435, 6,126,591, and so forth, for example.

### SUMMARY OF THE INVENTION

The endoscope device of the present invention comprises: an endoscope insert portion that comprises a solid-state image pickup element in the distal end portion thereof; a device main body that houses a signal processing device for processing an output signal from the solid-state image pickup element; an image recording section for recording a picture signal produced by the signal processing by the signal processing device; and an image display section for displaying the picture signal, wherein a general-purpose personal digital assistant is constituted by the image recording section and the image display section.

Further, the endoscope device of the present invention is an endoscope device, comprising: an endoscope main body, which comprises an elongated insert portion that is flexible and includes a solid-state image pickup element in the distal end portion thereof, an operation section provided at the back end of the insert portion, and a cable that extends from the operation section; a device main body, which comprises a connection portion for freely detachably connecting the cable of the endoscope main body, a signal processing device that processes an output signal from the solid-state image pickup element, and a light source for supplying illumination light to a light guide of the insert portion of the endoscope main body; an image recording section for recording a picture signal produced by the signal processing by the signal processing device; and an image display section for displaying the picture signal, wherein a general-purpose personal digital assistant is constituted by the image recording section and the image display section.

### BRIEF DESCRIPTION OF THE DRAWINGS

FIG. 1 is a block diagram showing the whole constitution of an endoscope device according to a first embodiment of the present invention;

FIG. 2 is a perspective view of the schematic external appearance shape thereof;

FIG. 3 is a perspective view of the external appearance of a modified example;

FIG. 4 is an illustrative view of thumbnail images displayed on a monitor;

FIG. 5 is a block diagram showing the whole constitution of an endoscope device according to a second embodiment of the present invention;

FIG. 6 is an illustrative view of the schematic external appearance shape thereof in an application example;

FIG. 7 is a block diagram showing the constitution of the device main body of the modified example; and

FIG. 8 is an illustrative view of the schematic constitution of an endoscope device according to a third embodiment of the present invention.

### DESCRIPTION OF THE PREFERRED EMBODIMENTS

Embodiments of the present invention will now be described with reference to the drawings.

(First Embodiment)

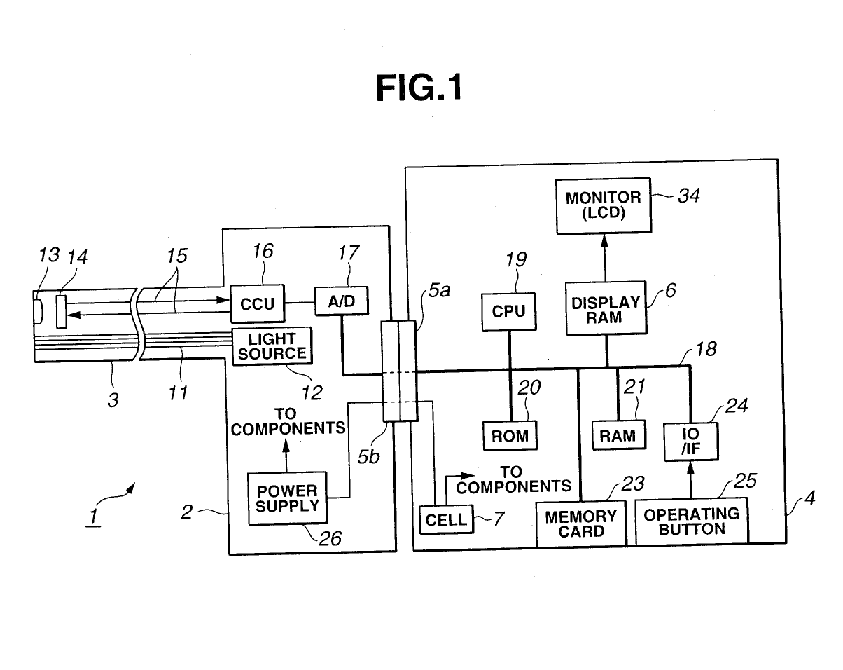

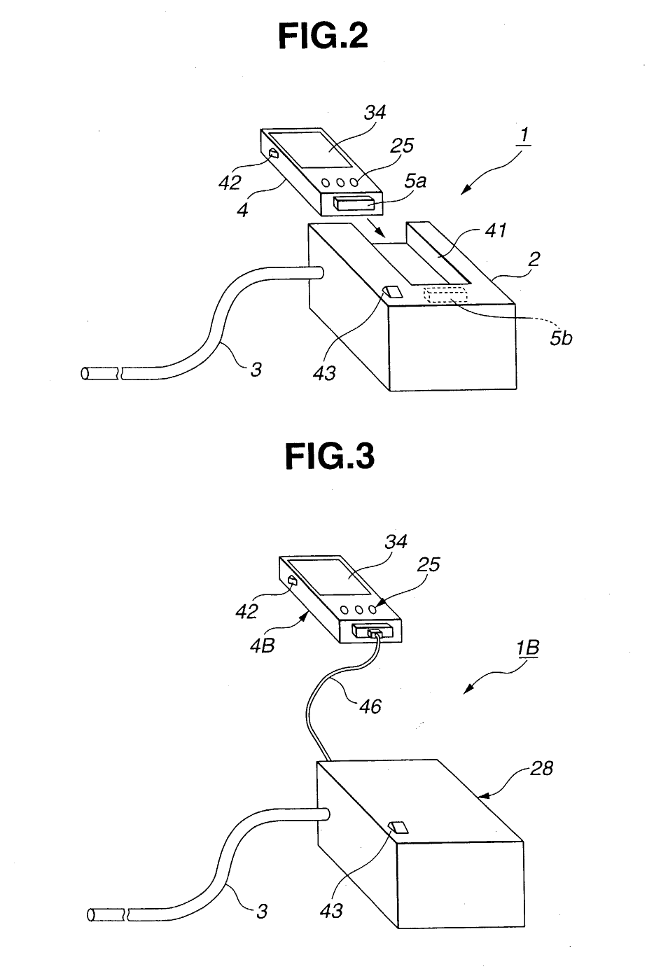

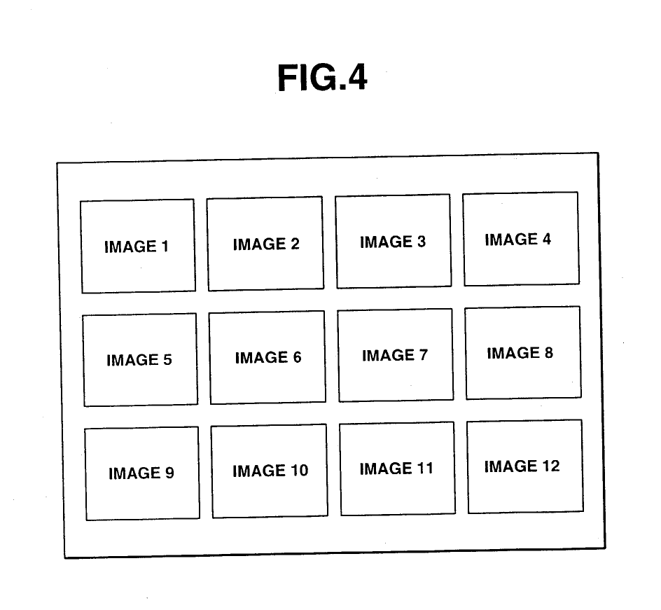

FIGS. 1 to 4 relate to the first embodiment of the present invention. FIG. 1 shows the whole constitution of the endoscope device according to the first embodiment; FIG. 2 shows the schematic external appearance shape thereof; FIG. 3 shows the external appearance shape of the modified example; FIG. 4 is an illustrative view showing thumbnail images displayed on a monitor.

As shown in FIG. 1, an endoscope device 1 according to the first embodiment of the present invention is constituted by an endoscope device main body (abbreviated to simply ‘device main body’ hereinafter) 2, a flexible and elongated insert portion 3 that extends from the device main body 2, and a personal digital assistant (abbreviated to ‘PDA’ hereinafter) 4 that is a general-purpose personal digital assistant connected to the device main body 2 via a connector 5.

A light guide 11 for transmitting illumination light passes through the inside of the insert portion 3, and transmits illumination light from a light source 12 housed in the device main body 2 to the light guide 11 so that this light exits via the surface of the distal end of the insert portion 3. Then, the emitted illumination light disperses forward from the surface of the distal end of the light guide 11 so as to illuminate a photographic subject that is an examination target such as an internal part of a plant, for example.

Further, an objective lens 13 is provided at an observation window of the distal end portion of the insert portion 3. A solid-state image pickup element 14 that comprises a CCD (Charge coupled device) and the like is disposed at the image formation position of the objective lens 13 such that the optical image of the photographic subject whose image is formed is photoelectrically (O/E) converted. The solid-state image pickup element 14 is connected via signal lines 15 to a CCU (Camera Control Unit) 16 which is a signal processing device housed in the device main body 2. The CCU 16 reads the O/E-converted image pickup signal (image signal) by applying a drive signal to the solid-state image pickup element 14, and subjects the image pickup signal thus read to processing that generates a picture signal (video signal).

The device main body 2 which is connected at the proximal end of the insert portion 3 comprises the light source 12 for supplying illumination light to the light guide 11, and the CCU 16 which is electrically connected to the solid-state image pickup element 14 by means of the signal lines 15 as described above. In addition, the device main body 2 houses an A/D converter 17 that converts the video signal outputted by the CCU 16 into a digital video signal, and a power supply 26 for supplying electric power to the light source 12 and the like. Further, a connector portion 5b of the PDA 4 is detachably connected to a connector portion 5a of the device main body 2.

The PDA 4 houses a busline 18, and, connected to this busline 18, are: a CPU (Central Processing Unit) 19 for performing control, a ROM (Read Only Memory) 20 that stores programs which can be controlled by the CPU 19, a RAM (Random Access Memory) 21 which is used by the CPU 19 as a work region, and so forth, a display RAM 6, a memory card 23 for recording image data, and an I/O interface 24. An operating button 25 is connected to the I/O interface (abbreviated to ‘IO/IF’ in the drawings) 24.

The busline 18 is also connected to the A/D converter 17 of the device main body 2 via connector portions 5b and 5a.

Furthermore, a monitor 34 in the form of a liquid crystal display and the like is connected to the display RAM 6. The CPU 19 writes output image data to the display RAM 6. The image data written to the display RAM 6 is always transmitted to the monitor 34 so as to be displayed thereby.

Further, the cell 7 housed in the PDA 4 supplies operational electric power to each of the parts in the PDA 4, and is also connected to the power supply 26 in the device main body 2 via the connector portions 5b and 5a. The cell 7 of the PDA 4 is thus supplied with electrical energy (that is, recharged) by the power supply 26 in the device main body 2.

One PDA 4 is constituted by the parts of the endoscope device 1 of this constitution except the insert portion 3, the light source 12, the CCU 16, and the device main body 2 that houses the A/D converter 17 and the power supply 26, and even the monitor 34 is contained as a part of the PDA 4.

The first function of the CPU 19 of the PDA 4 is to read I/O image data on the busline 18 and to transmit this data as output image data on the busline 18 as is. Further, the device main body 2 is provided with the operating button 25 such that when an operator operates the operating button 25, the operating signal is read from the I/O interface 24 to the CPU 19 via the busline 18.

For example, when the image record operation of the operating button 25 is executed, the CPU 19 performs a second function, namely that of writing the I/O image data to a predetermined region of the memory card 23. At the time of the image record operation, observed images are accordingly recorded successively on the memory card 23. The memory card 23 is freely detachable from the device main body 2, and permits data removal and storage, and reading of image data when connected to a personal computer or the like.

When the image playback operation is performed, the CPU 19 performs a third function, namely that of reading image data selected from a plurality of image data recorded on the memory card 23 and displaying this image data on the monitor 34.

In this case, for example, a plurality of image data which are recorded on the memory card 23 may be read out and reduced so as to create an overall single image (thumbnail images). Here, the CPU 19 transmits these thumbnail images as output image data. The thumbnail images shown in FIG. 4 (images 1 to 12 in FIG. 4) are displayed on the monitor 43. Next, when an image selection operation is performed by means of the operating button 25, the CPU 19 transmits the selected single image data as is. The selected image is then displayed on the monitor 34.

The functions of the CPU 19 are performed in accordance with programs which are pre-recorded on the ROM 20 as described above. Further, the RAM 21 is used as a work region for executing these functions.

FIG. 2 shows the schematic external appearance shape of FIG. 1.

As shown in FIG. 2, the endoscope insert portion 3 extends from the box-shaped device main body 2, a substantially rectangular receiving groove 41 is formed on the upper face, for example, of the device main body 2, and the connector portion 5b is provided in the innermost section of the receiving groove 41, for example.

Further, due to the PDA 4, which is provided with the connector portion 5a, being received by the receiving groove 41 so as to mate with the back portion thereof which is substantially rectangular, for example, the PDA 4 can be mechanically secured to the device main body 2 and also electrically connected thereto by attaching the connector portion 5a to the connector portion 5b.

The section of the monitor 34 constituted by a liquid crystal display and the operating button 25 in a section below the monitor 34 are formed on the upper face of the PDA 4. By operating the operating button 25, the above-described image record operation and recorded image playback operation, and so forth, may be performed.

Also, a power supply switch 42 is provided on a side of the PDA 4, and when this power supply switch 42 is mounted in the receiving groove 41, the power supply switch 42 can be set to an ON state by means of a protrusion or the like (not shown) provided on the wall face of the receiving groove 41 of the device main body 2.

Further, a power supply switch 43, which performs an ON/OFF operation in cooperation with the power supply of the device main body 2 and the PDA 4, is provided on the upper face or the like of the device main body 2. Due to this power supply switch 43, the light source 12 and the CCU 16 of the device main body 2, and the CPU 19 of the PDA 4, and the like, can enter an operational state, and the supply of electric power can be interrupted.

The endoscope device 1 of this embodiment with this constitution is characterized by being constituted by the insert portion 3, which performs endoscope examinations, by the device main body 2, which is connected to the insert portion 3 and houses the light source 12, the CCU 16, and the A/D converter 17, and by the general-purpose PDA 4, which is attached freely detachably to the device main body 2 and comprises image recording means and image display means.

Further, in addition to the program data for operation performed by the PDA 4 alone, another program data is written in the ROM 20 of the PDA 4, that program data allows the PDA 4 to function as the endoscope device 1 when the PDA 4 is mounted on the device main body 2.

In other words, application programs are installed on the PDA 4 so that a variety of applications such as memo software, schedule management software, and mail software run on the PDA basic operating system. However, programs are installed such that when the PDA 4 is also connected to the device main body 2, the CPU 19 performs a control operation to capture input image data outputted from the A/D converter 17 of the device main body 2 and to store this input image data in the display RAM 6, as well as an image record control operation in a case where the image record instruction button of the operating button 25 is operated, and an image playback control operation in a case where the image playback instruction button is operated.

Also, when an instruction button for image recording and the like is not provided on the PDA 4 in a unit, a program, which serves to allocate operating button functions so as to specify the operation of a specified button or a plurality of buttons as an instruction button for image recording or the like, is also installed.

The action of this embodiment with this constitution will now be described.

As shown in FIG. 2, according to this embodiment, the PDA 4 can be mounted on the device main body 2 by inserting the PDA 4 in the receiving groove 41 of the device main body 2. Further, the PDA 4 can be set to a state permitting an endoscope examination to be conducted by turning ON the power supply switch 43.

Accordingly, by inserting the endoscope insert portion 3 in an internal part of a plant, or the like, a photographic subject such as a plant internal part into which the insert portion 3 is inserted can be illuminated by the light source 12, and an image of the photographic subject thus illuminated is formed on the solid-state image pickup element 14 by the objective lens 13 and then O/E converted by the solid-state image pickup element 14.

The solid-state image pickup element 14 is driven by the CCU 16 in the device main body 2, and the image pickup signal outputted from the solid-state image pickup element 14 is subjected to signal processing, whereby a video signal is generated, then converted into a digital video signal by the A/D converter 17 and transmitted to the PDA 4 via the connector portions 5a and 5b.

In the PDA 4, the CPU 19 performs control such that the digital video signal inputted via the busline 18 from the A/D converter 17 side, that is, the input image data is written to the display RAM 6.

Further, the image data written to the display RAM 6 is continuously read out and outputted to the monitor 34 so that the image picked up by the image pickup element 14 is displayed on the display screen.

Further, as a result of the operator operating the operating button 25, an operating signal is read from the I/O interface 24 to the CPU 19 via the busline 18. For example, when the image record operation of the operating button 25 is performed, the CPU 19 writes I/O image data to a predetermined region of the memory card 23. At the time of the image record operation, observed images are accordingly recorded successively on the memory card 23. The memory card 23 is detachable from the PDA 4, and permits data removal and storage, and reading of image data when connected to a personal computer or the like.

In addition, when the image playback operation is performed, the CPU 19 reads out and plays back image data recorded on the memory card 23. In the case of image playback, the following variety of methods may be adopted, for example. First, the CPU 19 reads out a plurality of image data recorded on the memory card 23 and reduces this image data to create an overall single image (thumbnail images). The CPU 19 transmits these thumbnail images as output image data. The thumbnail images shown in FIG. 4 are displayed on the monitor. Next, when an image selection operation is performed by means of the operating button 25, the CPU 19 transmits the selected single image data as is. The selected image is then displayed on the monitor 34.

According to the present embodiment with this constitution and action, because the image recording means and image display means are constituted by a general-purpose PDA 4, manufacturing costs for the endoscope device 1 can be markedly reduced.

In other words, the PDA 4 can be produced at an unusually low price by means of mass production and therefore the development costs and product unit price and so forth can be markedly reduced.

Further, where the PDA 4 is concerned, the degree of integration of the internal parts for mass production is unusually high, and these parts can be reduced in size and made more lightweight. Therefore, the device main body part 2 can also be reduced in size and made more lightweight, and hence, it is possible to facilitate the endoscope examination by carrying it around and to make endoscope examinations straightforward also for examination target equipment and so forth in other fields.

Furthermore, according to the present embodiment, by attaching a communication card (not shown) to the part PDA 4, electronic mail and the Internet and the like can be utilized, recorded image data can be transmitted, and image data can be received.

Further, after an endoscope examination, the state of an examination target can be examined in detail by removing the part PDA 4 from the device main body 2 and playing back the images recorded by the PDA 4, and so forth. When the PDA 4 is connected to a personal computer, the images can be enlarged further and easily checked in detail on a display screen with a higher resolution, and the like.

Further, according to the present embodiment, a case of a PDA 4 that comprises the memory card 23 is shown. However, in the case of a PDA 4 that does not comprise the memory card 23, image data could be recorded on the RAM 21.

Further, according to the present embodiment, the PDA 4 is constituted so as to be detachable but a structure in which the PDA 4 is integrally incorporated in the device main body 2 is also possible. In this case also, the PDA 4 can be miniaturized, made more lightweight, and reduced in cost.

FIG. 3 shows the schematic constitution of an endoscope device 1B of a modified example. According to the endoscope device 1B, a device main body 2B connected to the insert portion 3 is constituted so as to be connected to a PDA 4B by means of a USB (Universal Serial Bus) cable 46, for example.

In this case, the device main body 2B is constituted such that, in the device main body 2 of FIG. 1, the A/D converter 17 is connected to a USB connector via a USB interface, and the PDA 4B is also constituted such that, in the PDA 4 of FIG. 1, there is a connection to a USB connector via a USB interface. Also, the endoscope device 1B is formed by connecting the USB cable 46 to the USB connectors.

Further, in a case where the PDA 4B is employed as the endoscope device 1B by connecting the device main body 2B and the USB cable 46, the USB cable 46, and so forth, is connected beforehand to install a program in the PDA 4B to enable it function as the endoscope device 1B.

In this case, the ROM 20 for storing the programs in the PDA 4B is constituted by a flash memory or EEPROM that permits simple electrical rewriting and that possesses nonvolatile characteristics. Further, depending on requirements, functional changes and program upgrades can also be easily implemented.

Otherwise, the constitution is the same as that for the first embodiment. Where the present modified example is concerned, as long as a PDA comprises a USB connector, even a PDA whose shape and functions and the like differ can be employed as the PDA 4B that constitutes the endoscope device 1B.

Therefore, in this case, the user is able to use a PDA 4B which is considered by the user to be easy to use, or a PDA 4B which the user is accustomed to using, by connecting the PDA 4B to the device main body 2B via the USB cable 46, whereby operability can be improved.

Further, in a case where the insert portion 3 is provided with a bending portion as described in the next embodiment, an operation section that bends this bending portion may be provided on the device main body 2 or the device main body 2B.

(Second Embodiment)

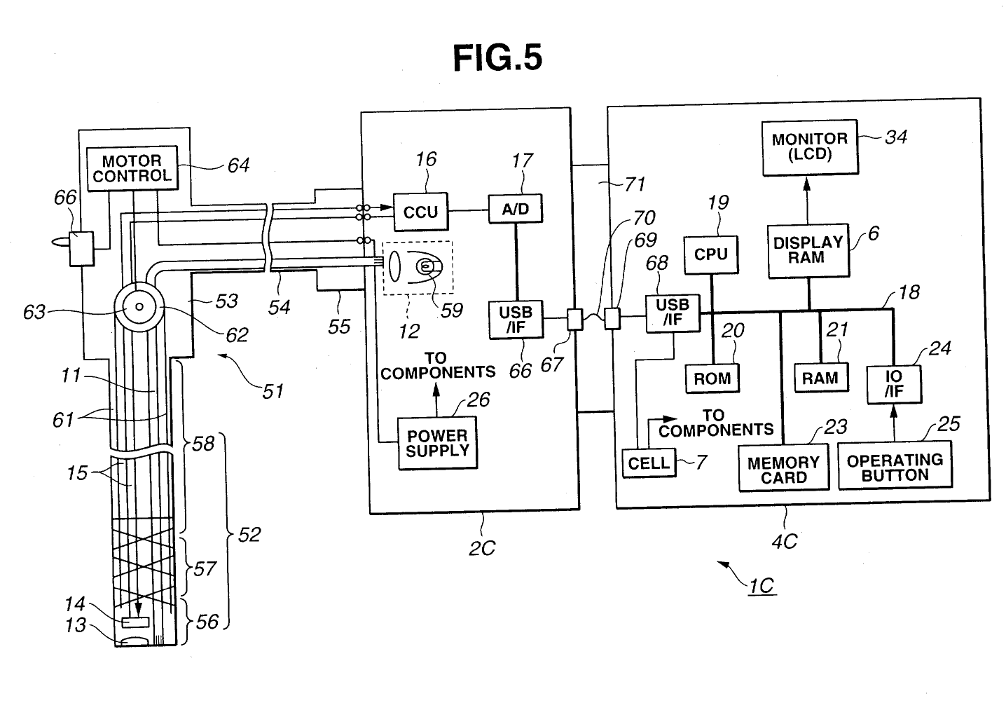

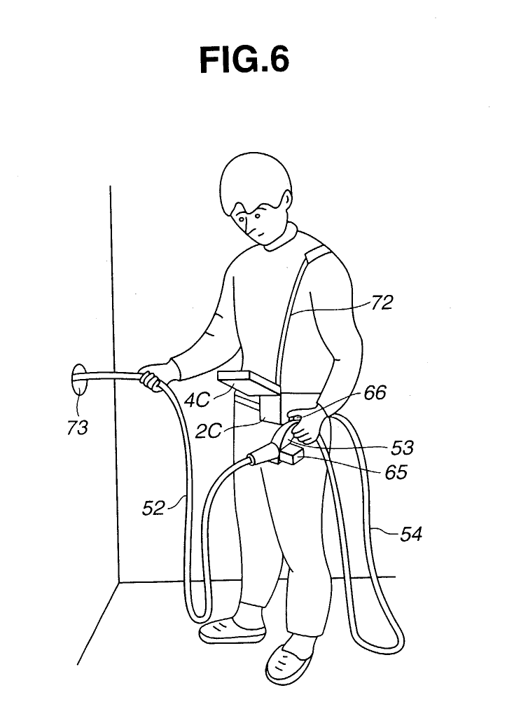

The second embodiment of the present invention will be described next with reference to FIGS. 5 and 6. FIG. 5 shows the whole constitution of an endoscope device 1C of the second embodiment, and FIG. 6 shows an external appearance thereof in an application example.

The endoscope device 1C shown in FIG. 5 is constituted by an endoscope main body 51, by a device main body 2C which is connected to the endoscope main body 51 and which houses the light source 12 and the CCU 16, and the like, and by a PDA 4C which is mounted on the device main body 2C so as to be capable of freely swinging.

The endoscope main body 51 comprises an elongated insert portion 52 that is flexible; an operation section 53 provided at the back end of the insert portion 52, and a universal cable 54 that extends from the operation section 53, wherein a connector 55 at the back end of the universal cable 54 is freely detachably connected to the device main body 2C.

The insert portion 52 comprises a hard distal end portion 56 provided at the distal end thereof, a bendable bending portion 57 provided at the back end of the distal end portion 56, and a flexible portion 58 that is long and flexible extending from the back end of the bending portion 57 to the operation section 53.

The light guide 11 passes through the inside of the insert portion 52 as described above. The back end of the light guide 11 is passed through by the operation section 53 and the universal cable 54 and thus protrude from the connector 55, and is then connected to the device main body 2C. As a result, the illumination light of a lamp 59 that constitutes the light source 12 is condensed by a condensing lens and supplied to the end portion of the light guide 11.

Further, the objective lens 13 and the solid-state image pickup element 14 disposed at the image formation position of the objective lens 13 are provided in the distal end portion 56 of the insert portion. The solid-state image pickup element 14 is connected to the connection point of the connector 55 via signal cables 15 that pass through the inside of the insert portion 52, the operation section 53, and the universal cable 54. By connecting the connector 55 to the device main body 2C, the solid-state image pickup element 14 is electrically connected to the CCU 16.

Further, a pair of wires 61 for transmitting the drive power for bending the bending portion 57 which is formed by linking a plurality of bending pieces such that same are capable of freely swinging passes through the insert portion 52. The distal ends of this pair of wires 61 are secured to the distal end portion 56, and the back ends of these wires are fixed so as to be wound around a pulley 62 within the operation section 53.

The rotating shaft of an electric bending motor 63 is attached to the central axis of the pulley 62, and the rotation of the motor 63 causes one of the pair of wires 61 to be pulled, while the other is relaxed, such that the bending portion 57 can be bent toward the pulled side.

The rotation of the motor 63 is controlled by a motor control circuit 64 provided in the operation section 53, for example. Also, the motor control circuit 64 is connected to a joystick 66 that constitutes bending operation means which are provided so as to extend from the operation section 53. The motor control circuit 64 controls the rotation of the motor 63 to the direction in which the joystick 66 is inclined.

Further, FIG. 5 shows the pair of wires 61 and one motor 63 only. However, the bending portion 57 can be bent in an optional direction such as upward and downward, left and right, and so forth, by using two pairs of wires and two motors.

Also, the motor 63 is housed within a motor box 65 that protrudes from an operation section 53 as shown in FIG. 6.

Further, the motor control circuit 64 is constituted such that operational electric power is supplied from the power supply 26 of the device main body 2C via power supply lines.

In the device main body 2C, a USB interface 66 is connected to the A/D converter 17 that is connected to the CCU 16, and the USB interface 66 is connected to a USB connector 67 whose connection point is exposed at the outer face of the device main body 2C.

Further, in the PDA 4 of FIG. 1, the PDA 4C further comprises a USB interface 68 which is connected to the busline 18. The USB interface 68 is connected to a USB connector 69 whose connection point is exposed at the outer face of the PDA 4C.

Also, the USB connectors 67 and 69 can be connected by a USB cable 70. Further, a hinge 71 that is capable of freely swinging is mounted on one side part of the device main body 2C and to one side part of the PDA 4C, whereby the PDA 4C is mounted so as to be capable of freely swinging with respect to the device main body 2C.

In this case, the surface of the PDA 4C in which the monitor 34 is provided is thus capable of freely swinging so as to face the upper face of the device main body 2C.

Further, the USB interface 68 is also connected to the cell 7 so that the cell 7 can also be recharged.

As described in FIG. 3, a program is pre-installed on the PDA 4C so as to function as the endoscope device 1C when connected to the device main body 2C.

An application example of the present embodiment with this constitution is shown in FIG. 5. A shoulder belt 72 is attached to the device main body 2C, and the user is thus able to hold the device main body 2C by putting the belt 72 on his or her shoulder.

Further, when an examination hole 73 is provided in a wall part, by grasping the operation section 53 of the endoscope main body 51 with one hand and holding the insert portion 52 with the other hand, the user performing the endoscope examination inserts the distal end of the insert portion into the examination hole 73.

Furthermore, at the time of the insertion, in cases where an internal part is to be observed or examined, the monitor 34 of the PDA 4C can then be brought into view by freely swinging the PDA 4C, which is attached to the device main body 2C so as to be capable of freely swinging, by means of the hinge 71.

Further, the status of the internal part can be observed or examined by observing the image on the monitor 34. Also, when the insert portion is in the process of being inserted in a deep section, in cases where the internal part is bent as observed via the observed image, the insert portion can be inserted smoothly by operating the joystick 66 with the finger of the hand grasping the operation section 53 such that the insert portion is made to bend in the direction of the bend.

With this embodiment too, results like those for the first embodiment may be obtained. Furthermore, operability can be improved because a bending operation can be performed by the hand grasping the operation section.

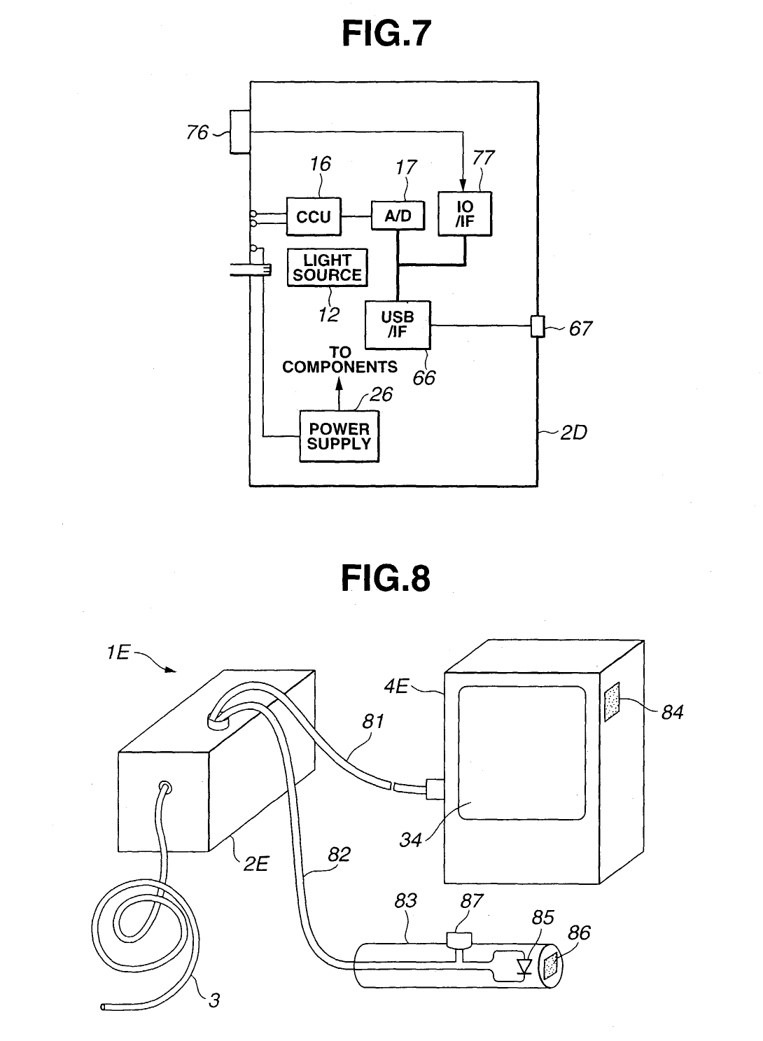

FIG. 7 shows a device main body 2D of a modified example.

In the device main body 2C of FIG. 5, the device main body 2D of this modified example further comprises a second operating button 76, and a second I/O interface 77 which is connected to the second operating button 76, the second I/O interface 77 being connected to the USB interface 66.

Further, image record and playback operations or other operations may be performed by means of the operating button 25 of the PDA 4C, and image record and playback operations and so forth may also be performed by operating the second operating button 76 of the device main body 2D, whereby operability is improved.

Otherwise, the constitution is the same as that for the second embodiment. Because this modified example permits image recording and so forth by both the PDA 4C and also the device main body 2D, the user is able to perform a straightforward operation by using either one.

Further, although, in this modified example, the device main body 2D is provided with the second operating button 76, this second operating button 76 may be provided on the operation section 53 of the endoscope main body 51. The second operating button 76 may be provided on the end of a cable extending from the device main body 2D, and a freeze button for issuing an instruction to display a still image may be provided on the operating button 25 or the second operating button 76.

(Third Embodiment)

FIG. 8 shows an endoscope device 1E according to the third embodiment of the present invention. The endoscope device 1E is constituted by a device main body 2E, the insert portion 3 that extends from the device main body 2E, a PDA 4E which is connected to the device main body 2E via a serial cable such as an RS232C (standard name of the Electrical Industry Association) cable 81 for example, and by an infrared light emission device 83 which is connected to the device main body 2E by means of a signal cable 82.

In the device main body 2B of FIG. 4, the device main body 2E contains an RS232C interface instead of the USB interface connected to the USB cable 46, and is constituted such that the built-in power supply 26 supplies electric power for light emission to the infrared light emission device 83 via a power supply cable 82.

Furthermore, in the PDA 4B of FIG. 3, the PDA 4E contains an RS232C interface instead of the USB interface connected to the USB cable 46, and contains an infrared receiving device.

The infrared receiving device comprises an infrared receiving window portion 84 in a side of the PDA 4E, for example, and further comprises a receiving device such as a receiving photodiode which is sensitive to infrared waves and is on the inside of a filter, that allows only infrared waves to pass, which is provided in the infrared receiving window portion 84, and comprises a wave shaping section for shaping a signal which has been O/E converted by the receiving device. The infrared receiving device sends the signal which has been thus shaped by the wave shaping section to the CPU 19, and, in accordance with this signal, the CPU 19 then performs image recording and an operation to playback a recorded image on the monitor 34.

Meanwhile, the infrared light emission device 83 contains an infrared light emitting diode 85 that emits infrared rays to the distal end side of a tubular body having a light blocking effect, for example, and infrared rays emitted by the infrared light emitting diode 85 pass through a filter which allows only infrared rays to pass and which is provided in an infrared emitting window portion 86, so that infrared rays can be emitted to the outside.

Further, the power supply cable 82 which is connected to the power supply 26 is provided with a switch portion 87 in series with the infrared light emitting diode 85. The switch portion 87 is provided with at least two or more plural switches (here, the two switches are SWI and SW2, for example), and the infrared light emitting diode 85 emits light with a light emission waveform that corresponds with the switch which is pushed.

In a state where the infrared emitting window portion 86 is made to face the infrared receiving window portion 84, when the switch SW1 is depressed, for example, the CPU 19 judges that an image record instruction signal is present and performs an image record operation.

Also, when the switch SW2 is depressed, the CPU 19 judges that an image playback instruction signal is present and performs an operation to play back an image recorded on the memory card 23.

According to the present embodiment, even in a case where the PDA 4E is disposed in a location separate from the device main body 2E, by grasping the infrared emission device 83 and operating the switch portion 87, an image record instruction and image playback instruction and so forth can be sent to the PDA 4E, and a remote instruction operation can be performed so as to execute an operation corresponding to the instruction operation carried out by the PDA 4E.

In addition, in this specification, a general-purpose personal digital assistant is described in terms of a PDA denoted by the reference numeral 4 and so forth. However, the general-purpose personal digital assistant is not limited to a PDA and is intended to refer to all small information terminal devices created based on the premise of handheld usage which are called Pocket PCs, Palm PCs, Handheld PCs, mobile PCs and so forth, for example.

Moreover, embodiments including those constituted through partial incorporation of the above-described embodiments also fall within the scope of the present invention.

Having described the preferred embodiments of the invention by referring to the accompanying drawings, it should be understood that the present invention is not limited to those precise embodiments and various changes and modifications thereof could be made by one skilled in the art without departing from the spirit or scope of the invention as defined in the appended claims.

What is claimed is:

1. An endoscope device, comprising:
an endoscope insert portion that comprises a solid-state image pickup element in the distal end portion thereof;
a device main body that houses a signal processing device for processing an output signal from the solid-state image pickup element;
an image recording section for recording a picture signal produced by the signal processing by the signal processing device; and
an image display section for displaying the picture signal,
wherein the image recording section and the image display section are constituted by a general-purpose personal digital assistant.

2. The endoscope device according to claim 1, wherein the device main body comprises a light source for supplying illumination light to a light guide of the endoscope insert portion.

3. The endoscope device according to claim 1, wherein the device main body has a built-in power supply, and, while electric power is supplied to the light source by the power supply, a cell housed in the personal digital assistant is recharged as a result of electrical energy being supplied to the cell.

4. The endoscope device according to claim 1, wherein the endoscope insert portion extends from the device main body.

5. The endoscope device according to claim 1, wherein the endoscope insert portion is freely detachably connected to the device main body.

6. The endoscope device according to claim 1,
wherein the device main body comprises an A/D converter that converts a picture signal, which is produced as a result of the output signal from the solid-state image pickup element being processed by the signal processing device, into a digital picture signal; and
the converted digital picture signal is supplied to a monitor and the image recording section via a busline housed in the general-purpose personal digital assistant which is connected to the device main body.

7. The endoscope device according to claim 1, wherein the personal digital assistant is separate from the device main body and is detachably connected to the device main body.

8. The endoscope device according to claim 1, wherein the personal digital assistant is integrally incorporated in the device main body,.

9. The endoscope device according to claim 7, wherein the personal digital assistant is detachably connected by a connector portion provided on the device main body and on the personal digital assistant respectively.

10. The endoscope device according to claim 7, wherein the personal digital assistant is detachably connected to the device main body via a connecting cable.

11. The endoscope device according to claim 10, wherein the personal digital assistant is detachably connected to the device main body by means of a USB cable.

12. The endoscope device according to claim 11, wherein the device main body and the personal digital assistant each comprises a USB interface.

13. The endoscope device according to claim 7, wherein the device main body comprises a receiving portion for receiving the information terminal that is connected to the device main body, and the receiving portion is provided with a device main body-side connecting portion.

14. The endoscope device according to claim 13, wherein the personal digital assistant further comprises a power supply switch and the power supply switch is set to an ON state when the personal digital assistant is received by the receiving portion of the device main body.

15. The endoscope device according to claim 1, wherein the personal digital assistant is provided with an instruction operation section.

16. The endoscope device according to claim 15, wherein an instruction operation section is also provided on the device main body-side.

17. The endoscope device according to claim 1, wherein the personal digital assistant is connected to the device main body and comprises a ROM for storing program data enabling the personal digital assistant to function as an endoscope device.

18. The endoscope according to claim 1, wherein, in a case where the general-purpose personal digital assistant is electrically connected to the device main body, software that affords the personal digital assistant the functions of an endoscope device when used in conjunction with the device main body can be installed on the personal digital assistant.

19. The endoscope device according to claim 1, wherein a communication section of the personal digital assistant is detachable.

20. The endoscope device according to claim 1, wherein the personal digital assistant is attached so as to be capable of freely swinging with respect to the device main body.

21. An endoscope device, comprising:
an endoscope main body, which comprises an elongated insert portion that is flexible and includes a solid-state image pickup element in the distal end portion thereof, an operation section provided at the back end of the insert portion, and a cable that extends from the operation section;
a device main body, which comprises a connection portion for freely detachably connecting the cable, a signal processing device that performs a signal processing on an output signal from the solid-state image pickup element, and a light source for supplying illumination light to a light guide of the insert portion of the endoscope main body;
an image recording section for recording a picture signal produced by the signal processing by the signal processing device; and
an image display section for displaying the picture signal,
wherein a general-purpose personal digital assistant is constituted by the image recording section and the image display section.

22. The endoscope device according to claim 21,
wherein the device main body further comprises an A/D converter that converts a picture signal, which is produced as a result of the output signal from the solid-state image pickup element being processed by the signal processing device, into a digital picture signal; and
the converted digital picture signal is supplied to a monitor and the image recording section via a busline housed in the general-purpose personal digital assistant which is connected to the device main body.

23. The endoscope device according to claim 21, wherein the personal digital assistant is separate from the device main body and is detachably connected to the device main body.

24. The endoscope device according to claim 21, wherein the personal digital assistant is detachably connected to the device main body via a connecting cable.

25. The endoscope device according to claim 24, wherein the personal digital assistant is detachably connected to the device main body by means of a USB cable, and the device main body and the personal digital assistant each comprises a USB interface.
